(19) United States
(12) Patent Application Publication
(10) Pub. No.: US 2012/0076754 A1
(43) Pub. Date: Mar. 29, 2012

(54) Use of IL-17 Polypeptides for Use in the Prevention or Treatment of Atherosclerosis

(76) Inventors: Ziad Mallat, Paris (FR); Alain Tedgui, Paris (FR); Soraya Taleb, Paris (FR)

(21) Appl. No.: 13/321,347

(22) PCT Filed: May 20, 2010
(86) PCT No.: PCT/EP2010/056950

§ 371 Date: Dec. 8, 2011

(30) Foreign Application Priority Data
May 20, 2009 (EP) 09305466.6

(51) Int. Cl. A61K 38/20; A61P 9/10; A61K 31/711

(52) U.S. Cl. 424/85.2; 514/44 R

(57) ABSTRACT

The present invention relates to the prevention or treatment of atherosclerosis, using an IL-17 polypeptide and pharmaceutical compositions thereof.

### FIELD OF THE INVENTION

The present invention relates to the prevention or treatment of atherosclerosis, and in particular to an IL-17 polypeptide for use in the prevention or treatment of atherosclerosis.

### BACKGROUND OF THE INVENTION

Atherosclerosis is the most common cause of death in western societies and is predicted to become the leading cause of cardiovascular disease in the world within two decades.

Atherosclerosis contributes to the development of atherosclerotic vascular diseases (AVD) which may affect the coronary arteries (causing ischemic heart disease), the cerebral circulation (causing cerebrovascular disease), the aorta (producing aneurysms that are prone to thrombosis and rupture) and peripheral blood vessels, typically the legs (causing peripheral vascular disease and intermittent claudication). Ischemic heart disease (IHD) includes angina (chest pain caused by insufficient blood supply to cardiac muscle) and myocardial infarction (death of cardiac muscle) and cerebrovascular disease includes stroke and transient ischemic attacks. One in three men and one in four women will die from IHD and the death rate for IHD was 58 per 100,000 in 1990.

Thus, there is a recognized and permanent need in the art for new reliable methods for treating atherosclerosis. The comprehension of new mechanisms and pathways can lead to new therapeutic targets and strategies.

Atherosclerosis is a chronic inflammatory disease of the vascular wall initiated in response to lipid infiltration and is actively controlled by an interplay between pathogenic Th1 and regulatory (Tr1, Th3 or Treg) immune responses. Recently, a novel Th subset has been identified producing high levels of IL17 (Th17). IL17 has been shown to promote several auto-immune diseases. However, its direct role in the control of atherosclerosis is still unexplored.

### SUMMARY OF THE INVENTION

The invention relates to an IL-17 polypeptide for use in the prevention or treatment of atherosclerosis.

The invention also relates to a pharmaceutical composition comprising an IL-17 polypeptide for the prevention or treatment of atherosclerosis.

### DETAILED DESCRIPTION OF THE INVENTION

The present invention relates to an IL-17 polypeptide for use in the prevention or treatment of atherosclerosis.

In the context of the invention, the term “treating” or “treatment”, as used herein, means reversing, alleviating, inhibiting the progress of, or preventing the disorder or condition to which such term applies, or one or more symptoms of such disorder or condition.

In its broadest meaning, the term “preventing” or “prevention” refers to preventing the disease or condition from occurring in a subject which has not yet been diagnosed as having it.

The term “patient” refers to any subject (preferably human) afflicted with or susceptible to be afflicted with atherosclerosis.

The method according to the present invention can be supplied to a patient, which has been diagnosed as presenting one of the following coronary disorders:
asymptomatic coronary artery coronary diseases with silent ischemia or without ischemia;
chronic ischemic disorders without myocardial necrosis, such as stable or effort angina pectoris;
acute ischemic disorders without myocardial necrosis, such as unstable angina pectoris;
ischemic disorders with myocardial necrosis, such as ST segment elevation myocardial infarction or non-ST segment elevation myocardial infarction.

A further aspect of the invention relates to a method for preventing a vascular or coronary disorder comprising the step of administrating a patient in need thereof with therapeutically effective amount of an IL-17 polypeptide.

In a particular embodiment, said coronary disorder or vascular disorders is selected from the group consisting of atherosclerotic vascular disease, such as aneurysm or stroke, asymptomatic coronary artery coronary diseases, chronic ischemic disorders without myocardial necrosis, such as stable or effort angina pectoris; acute ischemic disorders without myocardial necrosis, such as unstable angina pectoris; and ischemic disorders such as myocardial infarction.

The term “IL-17 polypeptide” has its general meaning in the art and includes naturally occurring IL-17 and conservative function variants and modified forms thereof. IL-17 is a family of structurally related cytokines. Representative examples of IL-17 cytokines include, but are not limited to, IL-17/IL17A, IL-17B, IL-17C, IL-17D, and IL-17F.

The IL-17 can be from any source, but typically is a mammalian (e.g., human and non-human primate) IL-17, and more particularly a human IL-17. The sequence of IL-17 protein and nucleic acids for encoding such proteins are well known to those of skill in the art. For example, Genbank Acc. No. AY460616 provides the complete DNA sequence of homo sapiens IL-17 gene; Genbank Acc. No. BD265544; Genbank Ace. No. BD265545; Genbank Acc. No. BD265546; Genbank Acc. No. BD265547; Genbank Acc. No. BD265548; Genbank Acc. No. BD265549; Genbank Acc. No. BD265550; Genbank Acc. No. BD265551; Genbank Acc. No. BD265552; Genbank Ace. No. BD265553; Genbank Acc. No. BD265554; Genbank Acc. No. BD265555; Genbank Acc. No. BD265556; Genbank Acc. No. BD265557, Genbank Acc. No. BD265558, Genbank Acc. No. BD265559, provide IL-17-associated mammalian cytokine sequences and polynucleotides encoding the same that are described in further detail in Japanese Patent No, 2002534122. The complete mRNA sequence of human IL-17 also is given at GenBank Acc. No. U32659 and is further described in Yao, Z. et al., 1995. However, it should be understood that, as those of skill in the art are aware of the sequence of these molecules, any IL-17 protein or gene sequence variant may be used as long as it has the properties of an IL-17 cytokine.

In addition to the numerous literature references describing the sequence of IL-17, incorporated herein by reference in their entirety are the teachings provided in U.S. Pat. No. 6,043,344, which describes human, rat and herpes virus herpes IL-17 proteins and nucleic acid compositions. SEQ ID NO:1 and SEQ ID NO:2 from U.S. Pat. No. 6,043,344 are particularly incorporated herein by reference as being the teaching of methods of making variants of these sequences taught in that patent and the methods of testing the compositions in various assays described therein. Also incorporated herein by reference is U.S. Pat. No. 6,074,849. U.S. Pat. No. 6,569,645 also is incorporated herein by reference as providing a teaching of polypeptides homologous to IL-17 and nucleic acid molecules encoding those polypeptides. Other variants of IL-17 that may be useful in the present application include the IL-17E polypeptides and IL-17E-encoding nucleic acids that are described in U.S. Pat. No. 6,579,520.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, most preferably at least 85%, and even more preferably at least 90%, and which has the same or substantially similar properties or functions as the native or parent protein to which it is compared.

In specific embodiments, it is contemplated that IL-17 polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 45 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the IL-17-derived proteins described herein for therapeutic delivery.

According to the invention, 11-17 polypeptide may be produced by conventional automated peptide synthesis methods or by recombinant expression. General principles for designing and making proteins are well known to those of skill in the art.

IL-17 polypeptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. Il-17 polypeptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art.

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. U.S. Pat. No. 6,569,645; U.S. Pat. No. 6,043,344; U.S. Pat. No. 6,074,849; and U.S. Pat. No. 6,579,520 provide specific examples for the recombinant production of IL-17 related proteins and these patents are expressly incorporated herein by reference for those teachings. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the IL-17-derived proteins of the invention, it would be necessary to employ vectors comprising polynucleotide molecules for encoding the IL-17-derived proteins. Methods of preparing such vectors as well as producing host cells transformed with such vectors are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan. Methods for the construction of mammalian expression vectors are disclosed, for example, in Okayama and Berg, 1983; Cosman et al., 1986; Cosman et al., 1984; EP-A-0367566; and WO 91/18982. Other considerations for producing expression vectors are detailed in e.g., Makrides et al., 1999; Kost et al., 1999. Wurm et al., 1999 is incorporated herein as teaching factors for consideration in the large-scale transient expression in mammalian cells for recombinant protein production.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (i.e., IL-17, a variant and the like). Thus, a promoter nucleotide sequence is operably linked to a given DNA sequence if the promoter nucleotide sequence directs the transcription of the sequence.

Similarly, the phrase “under transcriptional control” means that the promoter is in the correct location and orientation in relation to the nucleic acid to control RNA polymerase initiation and expression of the gene. Any promoter that will drive the expression of the nucleic acid may be used. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient to produce a recoverable yield of protein of interest. By employing a promoter with well known properties, the level and pattern of expression of the protein of interest following transfection or transformation can be optimized. Inducible promoters also may be used.

Another regulatory element that is used in protein expression is an enhancer. These are genetic elements that increase transcription from a promoter located at a distant position on the same molecule of DNA. Where an expression construct employs a cDNA insert, one will typically desire to include a polyadenylation signal sequence to effect proper polyadenylation of the gene transcript. Any polyadenylation signal sequence recognized by cells of the selected transgenic animal species is suitable for the practice of the invention, such as human or bovine growth hormone and SV40 polyadenylation signals.

Another aspect of the invention relates to a nucleic acid molecule encoding for an IL-17 polypeptide for use in the prevention or treatment of atherosclerosis.

Typically, said nucleic acid is a DNA or RNA molecule, which may be included in any suitable vector, such as a plasmid, cosmid, episome, artificial chromosome, phage or a viral vector as above described.

So, a further object of the invention relates to a vector comprising a nucleic acid encoding for an IL-17 polypeptide for use in the prevention or treatment of atherosclerosis.

A further object of the invention relates to a host cell comprising a nucleic acid encoding for an IL-17 polypeptide (or a vector comprising a nucleic acid thereof) for use in the prevention or treatment of atherosclerosis.

The present invention relates to a method for preventing or treating atherosclerosis in a patient in need thereof comprising the step of administrating said patient with therapeutically effective amount of IL-17 polypeptides (or nucleic acids encoding for IL-17 polypeptides or a vectors comprising a nucleic acids encoding for IL-17 polypeptides).

By a “therapeutically effective amount” is meant a sufficient amount of IL-17 polypeptides (or nucleic acid encoding for a IL-17 polypeptide or a vector comprising a nucleic acid encoding for a IL-17 polypeptide) to treat and/or to prevent atherosclerosis at a reasonable benefit/risk ratio applicable to any medical treatment.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

A further object of the invention relates to pharmaceutical compositions comprising an IL-17 polypeptide or a vector comprising a nucleic acid encoding for a IL-17 polypeptide) for the prevention or treatment of atherosclerosis.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The IL-17 polypeptide (or nucleic acid encoding for a IL-17 polypeptide or a vector comprising a nucleic acid encoding for a IL-17 polypeptide) may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The IL-17 polypeptide (or nucleic acid encoding for a IL-17 polypeptide or a vector comprising a nucleic acid encoding for a IL-17 polypeptide) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The IL-17 polypeptide (or nucleic acid encoding for an IL-17 polypeptide or a vector comprising a nucleic acid encoding for an IL-17 polypeptide) may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1. Quantitative analysis of the number of SOCS3-WT CD4⁺ cells that have adhered in vitro to IL1-activated murine endothelial cells, in the presence or absence of recombinant IL17 (r-IL17) or murine serum albumin used as control (ctr). Values represent mean values±s.e.m. of 3 experiments.

FIG. 2. Representative example of a Western blot showing reduction of IL1-induced VCAM-1 expression when murine endothelial cells are incubated with IL17.

FIG. 3. Quantitative analysis of atherosclerotic lesion size in the aortic root of 17-week old female chimeric Ldlr^{-/-} SOCS3-WT mice fed a high fat diet and treated with r-IL17 or control serum albumin for 5 weeks. Young animals were 12-week old.

FIG. 4. Quantitative analysis of lesional and adventitial T cell infiltration showing a clear reduction in T cell accumulation even in plaques of similar size.

FIG. 5. Quantitative analysis of endothelial VCAM-1 showing reduced expression in IL17-treated mice.

### EXAMPLE

Materials and Methods

Animals. We subjected 12- or 24-week old C57BL/6 Ldlr^{-/-} female mice to medullar aplasia by 9.5 gray lethal total body irradiation. We repopulated the mice with an intravenous injection of bone marrow cells isolated from femurs and tibias of female C57BL/6 SOCS3 wild type mice (SOCS3flox/flox) or mice with T cell-specific deletion in SOCS3 (SOCS3flox/floxick-Cre) generated by a conditional gene targeting approach using Cre-loxP system (Yasukawa, H. et al., 2003). After 4 weeks of recovery, mice were fed a high fat diet containing 15% fat, 1.25% cholesterol, and 0% cholate for 6 weeks. In some experiments, the reconstituted mice received an i.p. injection of either purified neutralizing anti-IL-17 antibody (200 mg/mouse, twice per week) (Uyttenhove, C. et al., 2006) or IgG control for 6 weeks. In others experiments using anti-IL17 antibody or IgG control, we injected i.p. the reconstituted SOCS3flox/flox:Lck-Cre mice with recombinant mouse IL-10 (eBioscience) diluted in PBS 0.05% mouse albumin (2 μg/mouse, twice per week) or PBS 0.05% albumin for 6 weeks. In another set of experiments, we treated i.p. 12- or 17-week old C57BL/6 Ldlr^{-/-} female mice with recombinant mouse IL-17A (eBioscience) diluted in PBS 0.05% mouse albumin (2 μg twice per week) or PBS 0.05% albumin during 5 weeks of high fat diet. Finally, six-week old male Apoe^{-/-}/Rag2^{-/-} mice were transferred intravenously with 3×10⁶ CD4⁺ cells recovered from either SOCS3-WT or SOCS3-overexpressing transgenic mice (Tg-SOCS3) (Seki, Y. et al., 2003), and put on high fat diet for 6 weeks. Experiments were conducted according to the French veterinary guidelines and those formulated by the European community for experimental animal use (L358-86/609EEC).

Extent and composition of atherosclerotic lesions. Quantification of lesion size and composition was performed as previously described (Taleb, S. et al., 2007). TUNEL staining was performed on fixed cryostat sections as previously described (Ait-Oufella, H. et al., 2007). Anti-mouse VCAM-1 antibody was obtained from BD Pharmingen. Tyrosine 705 phosphorylation of Stat3 was detected in the plaque using anti-phospho-Stat3 antibody (Cell Signaling). IL-17 staining in the plaque was performed using a validated anti-human/mouse IL17A antibody from Santa Cruz (Takahashi, N. et al., 2008). For sequential staining of P-Stat3, IL17 and CD3, sections stained with P-Stat3 or IL17 were destained using 1% hydrochloric acid in 70% ethanol (Kockx, M. M. et al., 1998) and LinBlock from Dako to remove all primary and secondary antibodies, and then stained for CD3. Controls included omitting the primary antibody against CD3, which resulted in negative staining for CD3, and use of serial sections that had not been previously stained by P-Stat3 or IL17 to verify that the staining was completely similar with the sections that were previously stained by P-Stat3 or IL17.

Cell recovery and purification, culture, proliferation and cytokine assays. CD11c⁺, CD4⁺CD25⁺ and CD4⁺CD25⁻ cells were purified and processed for cell proliferation assays and cytokine production as previously described in detail (Taleb, S. et al., 2007). IL-17 production in the supernatants was measured using specific ELISA (BD Biosciences and R&D Systems). Serum levels of IL-6, IL-27, IL-10 and TGF-β were measured using R&D Systems kits.

Adhesion assay. First, freshly isolated CD4⁺ cells were stimulated with anti-CD3 (1 μg/mL) for 1 hour. These cells were then stained during 30 minutes using a fluorescent probe (0.5 μM; CellTracker Orange CMTMR; Molecular Probes). Cell adhesion assay was performed on SVEC 4-10 cells (SVEC) (a simian virius 40-transformed mouse endothelial cell line). Briefly, SVEC cells were plated in 48-well plates in DMEM supplemented with 10% fetal calf serum and stimulated for 24 hours with 5 ng/mL of IL-1β (R&D Systems) prior to cell adhesion assay. After washout, fluorescent CD4⁺ cells (10⁵ per well) were made to adhere to SVECs for one hour. During adhesion assay, we added in certain experiments T cells in presence of anti-IL-17 blocking antibody (10 μg/mL) (Uyttenhove, C. et al., 2006) or an isotype-matched control, or with recombinant IL-17A (10 ng/mL) (eBiosciences) for 1 hour. In some experiments, SVECs were exposed to neutralizing antibody targeting VCAM-1 (50 μg/mL; R&D Systems) or its corresponding isotypic control for 1 hour before the cell adhesion protocol. After two more washouts, adherent lymphocytes were fixed in 4% paraformaldehyde and 5 different fields per well were counted under a fluorescence microscope (Zeiss microscope). Adhesion assay on isolated carotids was performed as previously described (Lemarie, C. A. et al., 2003). Briefly, the right and left primitive carotid arteries were isolated from C57BL/6 Ldlr^{-/-} and then cannulated at both extremities and immersed in an organ culture bath filled with DMEM with 5% fetal calf serum under sterile conditions in an incubator containing 5% CO2 at 37° C. Arterial segments were kept for 1 hour at an intra-luminal pressure of 80 mmHg for stabilization after surgery. Thereafter, vessels were injected with fluorescent CD4⁺ cells (5×10⁶ cells/mL) in presence of anti-IL-17 blocking antibody (10 μg/mL) or with its corresponding isotypic control, and allowed to adhere for one hour. After a 10-minute washout, vessels were fixed in 4% paraformaldehyde during 15 minutes, and placed on a microscope slide. Fluorescent adherent lymphocytes were counted under a fluorescence microscope.

Immunocytochemistry. After adhesion assay, SVEC cells plated on slides were fixed in 4% paraformaldehyde during 15 minutes followed by a treatment with 0.1% triton solution in order to permeabilize the cells. After 2 washouts with PBS, non-specific sites on SVECs were blocked with 10% serum. Thereafter, rabbit anti-phospho-p65 antibody (Rockland) was applied at a dilution of 1:50 for 1 hour at room temperature. Following two washes with PBS, the Alexafluor594 anti-rabbit antibody (Molecular Probes) was incubated (1:200) for one hour at room temperature. The slides were mounted with Fluoprep containing Dapi (Abcys) and visualized under a fluorescence microscopy.

Flow cytometry. Splenocytes were labeled with FITC-conjugated anti-CD4 (GK1.5 clone miltenyi) and intracellular Foxp3 staining was performed using PE-conjugated anti-mouse/rat Foxp3 (PFJK-16s, eBioscience) according to manufacturer's instructions (eBioscience). We also performed the staining of CD44 (Pgp-1, PharMingen). Anti-phospho-Stat3 staining was performed on CD4⁺ T cells stimulated with IL6 (100 ng/ml) during 15 min according to the manufacturer's protocol (Cell Signaling). Labeled cells were then analyzed by flow cytometry on an Epics XL flow cytometer (Beckman Coulter). IL17 and IL10 staining was performed using cytokine secretion assay kits (Miltenyi Biotch).

Western blot analysis. CD4⁺ cells were stimulated with coated anti-CD3 and anti-CD28 antibodies (2 μg/mL) for 60 min. Then, cells were lysed in detergent buffer containing protease inhibitor mixture (Roche) and sodium orthovanadate. Proteins were separated on 4-12% NuPage Tris-Bis gels using NuPage MES-SDS running buffer (Invitrogen) and then were transferred onto nitrocellulose membranes. The filters were probed with antibodies directed against phospho-Stat3 and Stat3 protein (Cell Signaling). On SVEC extracts, we quantified VCAM-1 expression by using specific antibody (R&D System). On human plaque extracts, phospho-Stat3, Stat3 and phospho-Stat1 staining was performed using specific antibodies purchased from Cell Signaling. Human IL-17 was detected using a monoclonal antibody (R&D Systems).

T cell-macrophage co-cultures. Macrophages were prepared from mouse bone marrow. Briefly, tibias and femurs of C57B1/6J mice were dissected, their marrow flushed out. Cells were grown for 7 to 10 days at 37° C. in RPMI 1640 medium, 20% FCS, and 20% macrophage-colony-stimulating factor (M-CSF)-rich L929-conditioned medium. Then, differentiated-macrophages were cultured either without T cells or with WT or SOCS3-cKO T cells for 24 h in the presence of soluble anti-CD3 antibody (1 ug/ml), after which the T cells were removed and the different cultures were washed twice with PBS and the macrophages were stimulated for 24 h with LPS (1 μg/ml) and IFN-γ (100 U/ml). In some experiments, SOCS3-WT or SOCS3-cKO T cells were incubated in presence of the neutralizing antibodies anti-IL10 (R&D Systems) (2 μg/mL) or anti-IL17 (10 μg/mL), or their corresponding isotype-matched controls. In other experiments, recombinant proteins IL10 and/or IL17 (10 ng/mL) (e Biosciences) were added on macrophages for 24 hr. On cells extracts, quantitative Real time PCR was performed to measure gene expression of M1 macrophage markers (NOS2, TNF-α) and M2 marker (Arginase-1). On supernatants, ELISAs were performed to measure IL-10 and IL-12 (BD biosciences) of the different conditions. TUNEL staining was performed on macrophages to analyze apoptosis.

Human carotid plaques. The processing and examination of the dissected atherosclerotic plaques has been described previously (Ho, G. H. et al., 1995; Verhoeven B. A., et al. 2004). Directly after excision, the atherosclerotic plaque is transferred to the laboratory. The segment with the greatest plaque burden (culprit lesion) is fixed in formaldehyde 4%, decalcified in ethylenediaminetetraacetic and embedded in paraffin. Segments adjacent to the culprit lesion are snap frozen in liquid nitrogen and stored at −80 Celsius for future analysis. The paraffin segment of the culprit lesion was cut on a microtome into sections of 5 μm for histological and immunohistochemical staining and the following stainings were performed to characterize the plaque: Picro Sirius red (collagen and lipid core), CD68 (macrophages), α-actin (smooth muscle cells), hematoxylin eosin (calcification, lipid core and intraplaque hemorrhage) and fibrin (intraplaque hemorrhage). All stainings were examined microscopically and plaque characteristics were scored semi-quantitatively as described previously (Verhoeven B. A., et al. 2004). Briefly, no or minor (categorized as ‘low’) macrophage and smooth muscle cell infiltration represents absent or minimal staining with few clustered cells, whereas moderate and heavy represent moderate or heavy staining with larger areas of clustered cells (categorized as ‘high’). The size of the lipid core was estimated as a percentage of total plaque area with a division in three categories: <10% (fibrous plaques), 10-40% (fibro-atheromatous plaques) and >40% (atheromatous plaques). Recently, we have demonstrated that our semi-quantitative analysis of atherosclerotic plaque histology is well reproducible, both intraobserver and interobserver (Hellings, W. E. et al., 2007).

Statistical analysis. Values are expressed as means±s.e.m. Differences between values were examined using nonparametric Mann-Whitney or Kruskal-Wallis tests and were considered significant at P<0.05.

Results

SOCS3 expression in T cells significantly impacts atherosclerotic lesion development. We first examined the effect of SOCS3 deletion in T cells on the development of atherosclerosis. We reconstituted low-density lipoprotein receptor-deficient mice (Ldlr^{-/-}) mice with either a wild type bone marrow from SOCS3flox/flox mice (the reconstituted mice are designated SOCS3-WT) or a bone marrow from mice with T cell-specific deletion in SOCS3 (SOCS3flox/flox:Lck-Cre) generated by a conditional gene targeting approach using Cre-loxP system (Kinjyo, I. et al., 2006; Yasukawa, H. et al., 2003) (designated SOCS3-cKO). Phosphorylated STAT3 was readily detectable in atherosclerotic lesions and we found a marked increase in phosphorylated-STAT3 in spleen-derived T cells from SOCS3-cKO mice compared with SOCS-WT mice, indicating an efficient and functional deletion of SOCS3 in T cells. We therefore quantified lesion size after 6 weeks on a high fat diet. We found an unexpected 50% reduction of aortic sinus atherosclerotic lesion size in SOCS3-cKO mice compared with controls, despite similar plasma cholesterol levels (18.4±0.7 vs 17.6±0.5 g/l, P=0.32). We observed a similar protection against atherosclerosis at the levels of the aortic sinus and the descending thoracic aorta in a separate set of old Ldlr^{-/-} mice reconstituted with SOCS3-cKO bone marrow compared with SOCS3-WT.

We then tested the effect of SOCS3 overexpression in T cells on the development of atherosclerosis. We reconstituted Apoe^{-/-}/Rag2^{-/-} mice with purified CD4⁺ cells recovered from either WT or SOCS3 transgenic (SOCS3-Tg) mice (18). As expected, we found reduced STAT3 phosphorylation in SOCS3-Tg T cells. After 6 weeks of high fat diet, spleen-derived CD4⁺ cells of mice transferred with CD4⁺ SOCS3-Tg cells showed reduced production of IL17 and IL10 but enhanced production of IL4 compared with T cells of mice transferred with CD4⁺ WT cells. This is consistent with previous studies that showed reduced Th17 and preferential Th2 differentiation of T cells isolated from SOCS3-Tg mice (Seki, Y. et al., 2003; Tanaka, K. et al., 2008). Interestingly, we found a 4-fold increase of lesion size in Apoe^{-/-}/Rag2^{-/-} mice transferred with CD4⁺ SOCS3-Tg cells compared with controls, despite similar plasma cholesterol levels (12.1±1.6 vs 14.1±0.78 g/l, P=0.25). Thus, SOCS3 expression in T cells significantly impacts atherosclerotic lesion development.

SOCS3 deletion in T cells enhances IL10 and IL17 production. We therefore examined potential athero-protective mechanisms related to SOCS3 deficiency. The reduction in atherosclerosis was associated with a significant decrease of T cell infiltration within the lesions, suggesting a modulation of the T cell phenotype. We found no difference in the number and suppressive function of natural CD4⁺CD25⁺Foxp3 regulatory T cells between the 2 groups of mice. Thus, we analyzed cytokine production by purified spleen-derived CD4⁺ cells in the presence of purified CD11c⁺ dendritic cells. We observed a significant reduction of IFN-γ production and an increase of IL10 by cells recovered from SOCS3-cKO mice, which was consistent with previous results showing preferential Th3/Tr1 differentiation and reduced Th1 polarization in mice lacking SOCS3 expression in T cells (Kinjyo, I. et al., 2006). However, we also detected a 3-fold increase in IL17 production by the purified SOCS3-deficient CD4⁺ cells compared with controls, consistent with the critical role of STAT3 activation in Th17 development (Chen, Z. et al., 2006; Dong, C., 2008). Flow cytometry analysis on freshly isolated cells showed no cells expressing both IL17 and IL10 suggesting that, in vivo, IL10 and IL17 are produced by distinct T cells, which is in agreement with a recent study showing that RORγ/t⁺ T cells producing both IL10 and IL17 could not be observed in vivo (Lochner, M. et al., 2008). Consistent with enhanced IL17 production, we found increased levels of circulating IL6 but reduced IL27 levels in the circulating blood of SOCS3-cKO mice. We also found increased production of IL6 by cultured SOCS3-cKO splenocytes after stimulation with anti-CD3. IL6 neutralization did not alter IL17 production, indicating that the Th17 profile of SOCS3-cKO cells was independent of IL6. Thus, our results indicate that in the absence of SOCS3 expression in T cells, there is a preferential switch towards increased production of both IL10 and IL17, which helps explain seemingly divergent previous results on Th3/Tr1 and Th17.

SOCS3 deletion in T cells induces an anti-inflammatory phenotype in macrophages. We therefore examined potential athero-protective mechanisms dependent on this particular T cell phenotype obtained after deletion of SOCS3. It was remarkable that the loss of SOCS3 in one cell type, the T cell, has produced significant effects on lesion development, where many other cell types are involved. Thus, we hypothesized that the T cell cytokine profile induced by SOCS3 deletion may modulate the inflammatory response of other cell types. In this regard, the innate immune system plays a critical role in atherogenesis (Hansson G. K., et al., 2006). Therefore, we examined the impact of SOCS3 deletion in T cells on macrophage infiltration and activation. The extent of lesional macrophage accumulation was not altered in SOCS3-cKO compared with SOCS3-WT mice (35022±6308 vs 36635±5223 μm², P=0.84). We hypothesized that this could be related to a differential modulation of macrophage survival within the lesions. Interestingly, we found a marked reduction in the size of the necrotic core in lesions of SOCS3-cKO mice. This was confirmed by the reduction of lesional TUNEL staining (data not shown) and a protection against macrophage apoptosis when bone marrow-derived macrophages were incubated in the presence of SOCS3-cKO CD4⁺ cells compared with SOCS3-WT CD4⁺ cells. Macrophage death within the lesions is highly promoted by pro-inflammatory signals. We found a significant reduction of IL12 and an increase in IL10 production by macrophages after co-incubation with SOCS3-cKO CD4⁺ cells compared to co-incubation with SOCS3-WT CD4⁺ cells. In addition, we found a significant reduction in the expression of NOS2 and TNF-α, and an increase of arginase-1 after co-incubation of macrophages with SOCS3-cKO T cells compared with SOCS3-WT cells, consistent with a limitation of M1 phenotype and the preservation of an anti-inflammatory potential. These effects persisted when T cells and macrophages were physically separated using trans-wells, suggesting the involvement of soluble factors. Neutralization of IL10 production and, to a lesser extent IL17, prevented some of the anti-inflammatory effects, whereas incubation with the recombinant cytokines was associated with an anti-inflammatory phenotype. Our results indicate that SOCS3 signaling in T cells not only impacts the T cell phenotype but also regulates the production of pro- and anti-inflammatory/atherogenic mediators by macrophages. This is consistent with a study showing that STAT3 activity in tumor cells mediates immune evasion through blockade of inflammatory signals production by the innate and adaptive immune system (Wang, T. et al. 2004).

Neutralization of IL17 abrogates athero-protection in mice with T cell-specific SOCS3 deletion and enhances vascular inflammation. The reduction of atherosclerosis in SOCS3-cKO mice was associated with a reduction in IFN-γ and an increase in both IL10 and IL17. The pro- and anti-atherogenic roles of IFN-γ and IL10, respectively, have been extensively addressed in previous studies (Tedgui, A. et al., 2006). However, the role of IL17 in atherosclerosis is still unexplored. Thus, we examined the direct role of IL17 production in the athero-protective effect of T cell-specific SOCS3 deletion. We generated additional series of chimeric Ldlr^{-/-} mice reconstituted with either SOCS3-cKO or SOCS3-WT bone marrow. The mice were put on a high fat diet to induce lesion formation and were treated with either a mouse monoclonal anti-IL17 neutralizing antibody (Uyttenhove, C. et al., 2006) or an isotype-matched control for 6 weeks. Again, we observed a significant reduction of lesion size in isotype-treated SOCS3-cKO mice compared with isotype-treated SOCS3-WT. Interestingly, neutralization of IL17 did not alter lesion size in SOCS3-WT mice, which are highly Th1 biased and produce low levels of IL17, but totally abrogated the athero-protective effect of T cell-specific SOCS3 deletion and led to a marked increase of lesion formation. Acceleration of lesion development was associated with a marked increase in vascular inflammation as revealed by increased VCAM-1 expression, marked T cell infiltration within the lesions and the adventitia of anti-IL17-treated SOCS3-cKO mice, and a switch toward reduced IL10 and increased IL4 production by spleen-derived CD4⁺ cells but no change in IFN-γ. Regulatory T cell function was not altered by anti-IL17 treatment and the pro-atherogenic effect of IL17 neutralization in SOCS3-cKO mice was not prevented by IL10 supplementation. These results identify an unprecedented role for SOCS3-controlled IL17 in the control of vascular inflammation and T cell accumulation within atherosclerotic lesions, which profoundly impacts on lesion development. Our results are in agreement with a study showing that IL17 neutralization was associated with enhanced T cell infiltration in a model of allergic asthma (Schnyder-Candrian, S. et al., 2006).

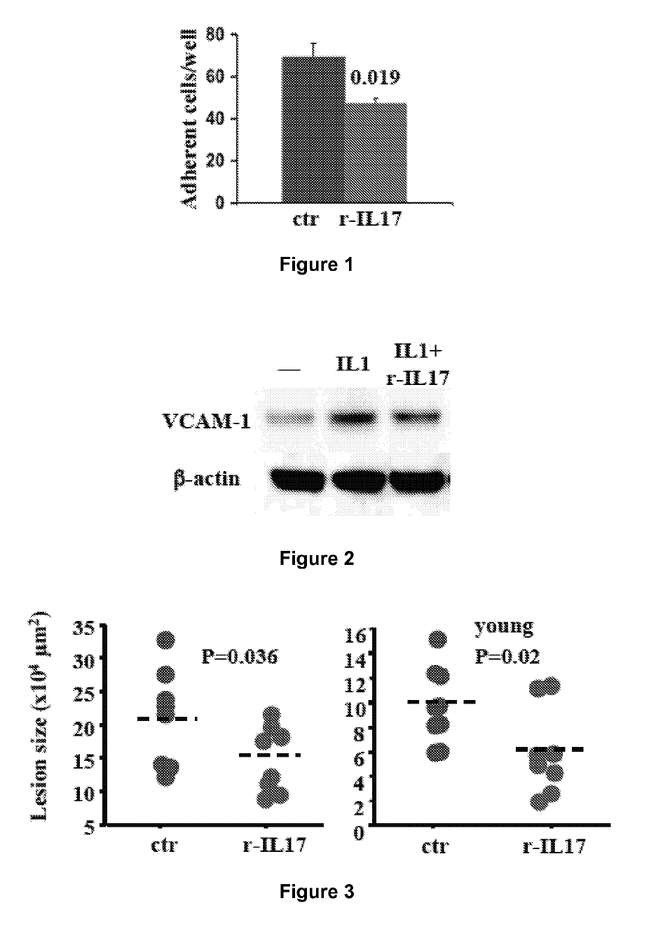

IL17 reduces endothelial VCAM-1 expression and T cell adhesion to vascular cells and carotid arteries. Since reduction of atherosclerosis in SOCS3-cKO mice was associated with a reduction in vascular T cell accumulation in vivo, and this was abrogated by IL17 neutralization, we examined the effect of SOCS3 expression on the vascular adhesive potential of activated T cells, which home more readily to inflammatory sites than naïve T cells. Adhesion assays were performed under static conditions since it is undisputed that atherosclerotic lesions preferentially develop in areas with very low or almost absent shear stress levels (Caro, C. G. et al., 1969). We found a significant reduction in ex-vivo adhesion of SOCS3-deficient T cells to carotid arteries retrieved from Ldlr^{-/-} mice, compared with control T cells (FIG. 2f). Similar results were obtained when adherence was tested on IL1-activated murine endothelial cells. Reduced adherence of SOCS3-deficient T cells was associated with reduced endothelial p65 phosphorylation and VCAM-1 expression, and neutralization of VCAM-1 significantly abrogated T cell adhesion. Interestingly, reduced adherence of SOCS3-deficient T cells to endothelial cells and carotid arteries was significantly prevented by IL17 neutralization. Supplementation with recombinant IL17 led to a significant reduction of the adhesion of control T cells to activated endothelium (FIG. 1), and to a reduction of IL1-induced endothelial VCAM-1 expression (FIG. 2). These results clearly support an important role of (SOCS3-controlled) IL17 production in the modulation of T cell recruitment and vascular inflammation.

In vivo administration of IL17 to Ldlr^{-/-} mice reduces endothelial VCAM-1 expression, vascular T cell infiltration and atherosclerotic lesion development.

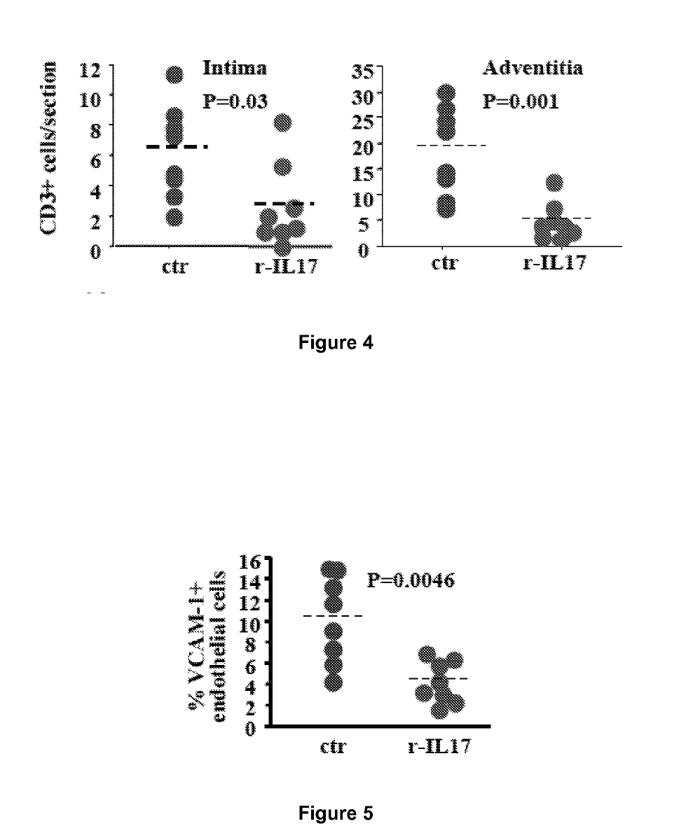

Neutralization of IL17 accelerated atherosclerosis in SOCS3-cKO mice but had no effect on lesion development in SOCS3-WT mice. We reasoned that this apparent discrepancy might be due to the low level of IL17 production by SOCS3-WT T cells (highly Th1 biased on a C57B1/6 background) and that supplementation of SOCS3-WT mice with IL17 might inhibit lesion development. Thus, we fed 17-week old female Ldlr^{-/-} mice a high fat diet and treated them with either recombinant IL17 (r-IL17) or control mouse serum albumin. Administration of r-IL17 led to a significant elevation of circulating IL17 levels and to moderate but significant elevation of IL6 production (comparable to levels detected in SOCS3-cKO mice), suggesting biological effects. We found no change in T cell cytokine profile or splenocyte inflammatory response in mice treated with r-IL17, indicating that, at this dosage, IL17 did not promote inflammatory cell activation. Interestingly, we found a significant reduction of atherosclerotic lesion development in mice supplemented with r-IL17 compared with controls (Figure=3). We reproduced the results in a different set of younger 12-week old female Ldlr^{-/-} mice (FIG. 3). Of note, athero-protection was associated with a marked inhibition of vascular inflammation as revealed by a significant inhibition of vascular T cell infiltration (FIG. 4) and a 60% reduction of endothelial VCAM-1 expression (FIG. 5). These results indicate that IL17 inhibits high fat-induced vascular inflammation and atherosclerotic lesion development.

Vascular expression of IL17 and P-Stat3 is associated with plaque stability. We then examined IL17 expression in mouse atherosclerotic arteries (Ldlr^{-/-} mice reconstituted with SOCS3-WT or SOCS3-cKO) using immuno-histochemistry. As expected, we were unable to detect IL17 expression in T cells of SOCS3-WT mice, and only occasional IL17-positive inflammatory cells were detected in lesions of SOCS3-cKO mice, which may be explained, at least in part, by the low number of activated SOCS3-deficient T cells that infiltrated the lesions. Unexpectedly, we found a marked staining of IL17 in medial smooth muscle cells of plaque-free areas (which was confirmed in normal arteries), and the staining appeared to be rapidly lost in medial smooth muscle cells underlying early lipid lesions. We confirmed the detection of IL17 in normal mouse aorta by Western blotting using a different antibody. These results show that C57B1/6 Ldlr^{-/-} mice, which are Th1-biased, produce low levels of IL17 in T cells and that vascular wall production of IL17 decreases during the development of atherosclerosis, suggesting a relationship between smooth muscle cell differentiation and IL17 expression.

Finally, we examined the relevance of our experimental findings to human atherosclerosis. We studied human carotid atherosclerotic arteries retrieved from patients undergoing carotid endarterectomy. Stat3 phosphorylation and IL17 expression were readily detectable in CD3-positive cells of human atherosclerotic plaques, although not all CD3⁺ cells expressed IL17 or phosphorylated Stat3. We could also detect Stat3 phosphorylation in other cell types, as expected. Interestingly, we also detected IL17 expression in vascular smooth muscle cells, and consistent with our data in mice, we found a clear decrease in its expression by medial smooth muscle cells underlying advanced atherosclerotic lesions. Interestingly, semi-quantitative assessment of Stat3 phosphorylation and IL17 expression using Western blot showed increased levels of these 2 markers in plaques with fibrous (stable) compared to atheromatous (unstable) phenotype. Consistent with this finding, increased levels of Stat3 phosphorylation were significantly associated with a low macrophage content, and increased levels of IL17 were associated with a lower macrophage infiltration and a higher smooth muscle cell content. No relation was found between Stat3 or P-Stat1 levels and plaque phenotype.

In conclusion, we show that endogenous expression of SOCS3 in T cells interrupts a major regulatory pathway in atherosclerosis through inhibition of IL17 production. Interestingly, patients have been identified with defective STAT3 signaling (Minegishi, Y. et al., 2007) and IL17 production (Milner, J. D. et al., 2008; Ma, C. S. et al., 2008), and recent observational studies reported the abnormal occurrence of vascular inflammation (Ling, J. C. et al., 2007; Van der Meer, J. W. et al., 2006) in this setting despite the absence of classical cardiovascular risk factors. Although the reported vascular abnormalities consisted mainly of inflammatory vascular aneurysms, some cases of coronary atherosclerosis have been reported in these young individuals (Freeman, A. F. et al., 2007). Thus, our results may have important implications for the understanding of the pathophysiological mechanisms of vascular inflammation in humans and identify novel targets for disease modulation.

### REFERENCES

Throughout this application, various references describe the state of the art to which this invention pertains. The disclosures of these references are hereby incorporated by reference into the present disclosure.
Ait-Oufella, H., K. Kinugawa, J. Zoll, T. Simon, J. Boddaert, S. Heeneman, O. Blanc-Brude, V. Barateau, S. Potteaux, R. Merval, B. Esposito, E. Teissier, M. J. Daemen, G. Leseche, C. Boulanger, A. Tedgui, and Z. Mallat. 2007. Lactadherin-defciency induces apoptotic cell accumulation, alters the regulatory immune response, and accelerates atherosclerosis in mice. Circulation 115:2168-2177.
Babe et al., Biotechnol Genet Eng Rev.; 17:213-52, 2000
Caro, C. G., J. M. Fitz-Gerald, and R. C. Schroter. 1969. Arterial wall shear and distribution of early atheroma in man. Nature 223:1159-1160.
Chen, Z., A. Laurence, Y. Kanno, M. Pacher-Zavisin, B. M. Zhu, C. Tato, A. Yoshimura, L. Hennighausen, and J. J. O'Shea. 2006. Selective regulatory function of Socs3 in the formation of IL-17-secreting T cells. Proc Natl Acad Sci USA 103:8137-8142.
Colosimo A, Goncz K K, Holmes A R, Kunzelmann K, Novelli G, Malone R W, Bennett M J, Gruenert D C. 2000. Transfer and expression of foreign genes in mammalian cells. Biotechniques. 29(2):314-8. Dong, C. 2008. TH17 cells in development: an updated view of their molecular identity and genetic programming. Nat Rev Immunol 8:337-348.
Cosman D, Cerretti D P, Larsen A, Park L, March C, Dower S, Gillis S, Urdal D. 1984. Cloning, sequence and expression of human interleukin-2 receptor. Nature. 312(5996):768-71.
Cosman D, Wignall J, Lewis A, Alpert A, Cerretti D P, Park L, Dower S K, Gillis S, Urdal D L. 1986. High level stable expression of human interleukin-2 receptors in mouse cells generates only low affinity interleukin-2 binding sites. Mol Immunol. 23:935-41. Freeman, A. F., D. E. Kleiner, H. Nadiminti, J. Davis, M. Quezado, V. Anderson, J. M. Puck, and S. M. Holland. 2007. Causes of death in hyper-IgE syndrome. J Allergy Clin Immunol 119:1234-1240.
Giga-Hama Y, Kumagai H. 1999. Expression system for foreign genes using the fission yeast Schizosaccharomyces pombe. Biotechnol Appl Biochem. 30 (Pt 3):235-44.
Ghosh S, Parvez M K, Banerjee K, Sarin S K, Hasnain S E. 2002. Baculovirus as mammalian cell expression vector for gene therapy: an emerging strategy. Mol Ther. 6(1):5-11.
Gross and Meienhofer, eds, Academic Press, New York, 1-284, (1979),
Hansson, G. K., and P. Libby. 2006. The immune response in atherosclerosis: a double-edged sword. Nat Rev Immunol 6:508-519.
Hellings, W. E., G. Pasterkamp, A. Vollebregt, C. A. Seldenrijk, J. P. De Vries, E. Velema, D. P. De Kleijn, and F. L. Moll. 2007. Intraobserver and interobserver variability and spatial differences in histologic examination of carotid endarterectomy specimens. J Vasc Surg 46:1147-1154.
Ho, G. H., F. L. Moll, P. P. Joosten, E. D. van de Pavoordt, and T. T. Overtoom. 1995. The Mollring Cutter remote endarterectomy: preliminary experience with a new endovascular technique for treatment of occlusive superficial femoral artery disease. J Endovasc Surg 2:278-287.
Kaufman R J. 2000. Overview of vector design for mammalian gene expression. Mol Biotechnol. 16(2):151-60.
Kinjyo, I., H. Inoue, S. Hamann, S. Fukuyama, T. Yoshimura, K. Koga, H. Takaki, K. Himeno, G. Takaesu, T. Kobayashi, and A. Yoshimura. 2006. Loss of SOCS3 in T helper cells resulted in reduced immune responses and hyperproduction of interleukin 10 and transforming growth factor-beta 1. J Exp Med 203:1021-1031.
Kockx, M. M., J. Muhring, M. W. Knaapen, and G. R. de Meyer. 1998. RNA synthesis and splicing interferes with DNA in situ end labeling techniques used to detect apoptosis. Am J Pathol 152:885-888.
Kost T A, Condreay J P. 1999. Recombinant baculoviruses as expression vectors for insect and mammalian cells. Curr Opin Biotechnol. 10(5):428-33.
Lemarie, C. A., B. Esposito, A. Tedgui, and S. Lehoux. 2003. Pressure-induced vascular activation of nuclear factor-kappaB: role in cell survival. Circ Res 93:207-212.
Ling, J. C., A. F. Freeman, A. M. Gharib, A. E. Arai, R. J. Lederman, D. R. Rosing, and S. M. Holland. 2007. Coronary artery aneurysms in patients with hyper IgE recurrent infection syndrome. Clin Immunol 122:255-258.
Lochner, M., L. Peduto, M. Cherrier, S. Sawa, F. Langa, R. Varona, D. Riethmacher, M. Si-Tahar, J. P. Di Santo, and G. Eberl. 2008. In vivo equilibrium of proinflammatory IL-17+ and regulatory IL-10+Foxp3+RORgamma t+T cells. J Exp Med 205:1381-1393.
Ma, C. S., G. Y. Chew, N. Simpson, A. Priyadarshi, M. Wong, B. Grimbacher, D. A. Fulcher, S. G. Tangye, and M. C. Cook. 2008. Deficiency of Th17 cells in hyper IgE syndrome due to mutations in STAT3. J Exp Med 205:1551-1557.
Makrides S C. 1999. Components of vectors for gene transfer and expression in mammalian cells. Protein Expr Purif. 17(2):183-202.
Merrifield B. 1986. Solid phase synthesis. Science. 232(4748):341-7.
Milner, J. D., J. M. Brenchley, A. Laurence, A. F. Freeman, B. J. Hill, K. M. Elias, Y. Kanno, C. Spalding, H. Z. Elloumi, M. L. Paulson, J. Davis, A. Hsu, A. I. Asher, J. O'Shea, S. M. Holland, W. E. Paul, and D. C. Douek. 2008. Impaired T(H)17 cell differentiation in subjects with autosomal dominant hyper-IgE syndrome. Nature 452:773-776.
Minegishi, Y., M. Saito, S. Tsuchiya, I. Tsuge, H. Takada, T. Hara, N. Kawamura, T. Ariga, S. Pasic, O. Stojkovic, A. Metin, and H. Karasuyama. 2007. Dominant-negative mutations in the DNA-binding domain of STAT3 cause hyper-IgE syndrome. Nature 448:1058-1062.
Okayama H, Berg P. 1983. A cDNA cloning vector that permits expression of cDNA inserts in mammalian cells. Mol Cell Biol. 3(2):280-9.5chnyder-Candrian, S., D. Togbe, I. Couillin, I. Mercier, F. Brombacher, V. Quesniaux, F. Fossiez, B. Ryffel, and B. Schnyder. 2006. Interleukin-17 is a negative regulator of established allergic asthma. J Exp Med 203:2715-2725.
Seki, Y., H. Inoue, N. Nagata, K. Hayashi, S. Fukuyama, K. Matsumoto, O. Komine, S. Hamano, K. Himeno, K. Inagaki-Ohara, N. Cacalano, A. O'Garra, T. Oshida, H. Saito, J. A. Johnston, A. Yoshimura, and M. Kubo. 2003. SOCS-3 regulates onset and maintenance of T(H)2-mediated allergic responses. Nat Med 9:1047-1054.
Stewart and Young, Solid Phase Peptide Synthesis, 2d. ed., Pierce Chemical Co., (11984);
Taleb, S., O. Herbin, H. Ait-Oufella, W. Verreth, P. Gourdy, V. Barateau, R. Merval, B. Esposito, K. Clement, P. Holvoet, A. Tedgui, and Z. Mallat. 2007. Defective Leptin/Leptin Receptor Signaling Improves Regulatory T Cell Immune Response and Protects Mice From Atherosclerosis. Arterioscler Thromb Vasc Biol
Takahashi, N., I. Vanlaere, R. de Rycke, A. Cauwels, L. A. Joosten, E. Lubberts, W. B. van den Berg, and C. Libert. 2008. IL-17 produced by Paneth cells drives TNF-induced shock. J Exp Med 205:1755-1761.
Tam et al., J. Am. Chem. Soc., 105:6442, (1983);
Tanaka, K., K. Ichiyama, M. Hashimoto, H. Yoshida, T. Takimoto, G. Takaesu, T. Torisu, T. Hanada, H. Yasukawa, S. Fukuyama, H. Inoue, Y. Nakanishi, T. Kobayashi, and A. Yoshimura. 2008. Loss of suppressor of cytokine signaling 1 in helper T cells leads to defective Th17 differentiation by enhancing antagonistic effects of IFN-gamma on STAT3 and Smads. J Immunol 180:3746-3756.
Tedgui, A., and Z. Mallat. 2006. Cytokines in atherosclerosis: pathogenic and regulatory pathways. Physiol Rev 86:515-581.
Uyttenhove, C., and J. Van Snick. 2006. Development of an anti-IL-17A auto-vaccine that prevents experimental auto-immune encephalomyelitis. Eur J Immunol 36:2868-2874.
Van der meer, J. W., C. M. Weemaes, J. H. van Krieken, C. E. Blomjous, C. E. van Die, M. G. Netea, and S. J. Bredie. 2006. Critical aneurysmal dilatation of the thoracic aorta in young adolescents with variant hyperimmunoglobulin E syndrome. J Intern Med 259:615-618.
Verhoeven, B. A., E. Velema, A. H. Schoneveld, J. P. de Vries, P. de Bruin, C. A. Seldenrijk, D. P. de Kleijn, E. Busser, Y. van der Graaf, F. Moll, and G. Pasterkamp. 2004. Athero-express: differential atherosclerotic plaque expression of mRNA and protein in relation to cardiovascular events and patient characteristics. Rationale and design. Eur J Epidemiol 19:1127-1133.
Wang, T., G. Niu, M. Kortylewski, L. Burdelya, K. Shin, S. Zhang, R. Bhattacharya, D. Gabrilovich, R. Heller, D. Coppola, W. Dalton, R. Jove, D. Pardoll, and H. Yu. 2004. Regulation of the innate and adaptive immune responses by Stat-3 signaling in tumor cells. Nat Med 10:48-54.
Wurm F, Bernard A. 1999. Large-scale transient expression in mammalian cells for recombinant protein production. 1: Curr Opin Biotechnol. 1999. 10(2):156-9. Yao Z, Painter S L, Fanslow W C, Ulrich D, Macduff B M, Spriggs M K, Armitage R J. 1995. Human IL-17: a novel cytokine derived from T cells. J Immunol 155(12):5483-6.
Yasukawa, H., M. Ohishi, H. Mori, M. Murakami, T. Chinen, D. Aki, T. Hanada, K. Takeda, S. Akira, M. Hoshijima, T. Hirano, K. R. Chien, and A. Yoshimura. 2003. IL-6 induces an anti-inflammatory response in the absence of SOCS3 in macrophages. Nat Immunol 4:551-556.
Zalipsky S, Mullah N, Harding J A, Gittelman J, Guo L, DeFrees S A. 1997. Poly(ethylene glycol)-grafted liposomes with oligopeptide or oligosaccharide ligands appended to the termini of the polymer chains. Bioconjug Chem. 8(2):111-8.

1-4. (canceled)

5. A method for preventing or treating atherosclerosis in a subject in need thereof, comprising the step of
administering to said subject a pharmaceutical composition comprising
an IL-17 polypeptide; or
a nucleic acid encoding an IL-17 polypeptide;
in an amount sufficient to prevent or treat said atherosclerosis.

6. The method of claim 5, wherein said nucleic acid encoding an IL-17 polypeptide is present in a vector.

7. The method of claim 5, wherein said pharmaceutical composition comprises an IL-17 polypeptide.

8. The method of claim 5, wherein said pharmaceutical composition comprises a nucleic acid encoding an IL-17 polypeptide.
